(19) United States
(12) Patent Application Publication
(10) Pub. No.: US 2006/0198912 A1
(43) Pub. Date: Sep. 7, 2006

(54) Topical treatment compositions

(76) Inventor: Kuki Gallmann, Nairobi (KE)

Correspondence Address: BUTZEL LONG, 350 SOUTH MAIN STREET, SUITE 300, ANN ARBOR, MI, 48104, US

(21) Appl. No.: 11/282,087

(22) Filed: Nov. 17, 2005

Related U.S. Application Data
(60) Provisional application No. 60/628,612, filed on Nov. 17, 2004.

(51) Int. Cl. A61K 36/28

(52) U.S. Cl. 424/764

(57) ABSTRACT

A dermatological treatment composition that includes an oil extract of leleshwa. The oil extract of leleshwa can be used alone or combined together with other ingredients in liquid, ointment, salve, gel, cream or foam formulations to treat a variety of dermatological conditions, including athlete's foot, verruca, shingles, atopic dermatitis, and candida.

### RELATED APPLICATION

The present application is based upon U.S. Provisional Patent Application Ser. No. 60/628,612 entitled “Topical Treatment Compositions” filed Nov. 17, 2004 to which priority is claimed under 35 U.S.C. §120, the entire specification of which is hereby expressly incorporated herein by reference.

### TECHNICAL FIELD

The present invention relates to chemical compositions that are used to treat various dermatological conditions. More particularly, the present invention is directed to natural plant extracts that can be used alone or combined with other ingredients, and which improve dermatological conditions including various disorders and diseases.

### BACKGROUND ART

The Tarchonanthus camphoratus is a shrub that is widespread in Southern Africa. It grows in thickets of bushveld, grassland, forests and semi-desert areas. It thrives in sandy soils and is abundant in the Rift uplands area. Tarchonanthus camphorates is a semi-deciduous small tree or shrub that grows in large uniform groups, but grows larger and more densely when it grows alone among other trees in the bush.

Tarchonanthus camphorates is known by a number of different names such as wild cotton, sage wood, African wild sage, wildesalie, kamferhout, kamferbos, sieriehout, vaalbos, veld-vaalbos, vaaibos, sauto mofahlana, rolog e thaalaping mohathla, well known in the local Maasai dialect with the name of “leleshua” or “leleshwa,” (used herein) in the abissianian language “Ebok,” the Swahili dialect “mikalambati, and so on.

The Tarchonanthus camphoratus has regular branches with velutinous white-greenish oval or lanceolate shaped leaves ranging from 4 to 13 cm length having a strong camphorated aromatic smell, whence its Linneana denomination.

Traditionally, leleshwa has many uses. The leaves are crushed and applied to the skin for cuts and wounds. Leleshwa is also used as a natural insect repellant by many tribes in Africa. The Massai often use the soft aromatic leaves of leleshwa as bedding, which is said to promote deep sleep and deter insects. In the western Cape region a hot poultice of the leaves is applied to the chest to relieve asthma, bronchitis and chest inflammations.

When crushed, leleshwa leaves emit a strong camphoraceous scent. Many wild animals that live in the areas where leleshwa grows, particularly cape buffaloes and black rhinos, have been observed rubbing themselves against a leleshwa tree or shrub. Upon closer inspection, it is revealed that those animals, which have crushed the leaves onto their skin, were relatively free from ticks.

U.S. Pat. No. 5,607,674 to Rovesti et al. discloses the use of Tarchonanthus camphorate or leleshwa and its derivatives in formulations and compositions having insect repellant, anti-irritant, anti-edema, decongestant and soothing properties. Rovesti et al. teaches the use of the essential oils from leleshwa which can be obtained from extraction methods including distillation.

Allergic 2 Pets, LLC (New York, N.Y.) sells a product called Exema Cleanse™ for treating pet skin aliments. The active ingredient in Exema Cleanse™ is the essential oil from young leleshwa leaves which is stated to be an extremely effective antiseptic, antiviral, antibacterial, and antifungal and a natural detergent and disinfectant.

Essential Oil University, Inc (ww.essentialoils.org) sells leleshwa essential oil for reducing dandruff and reducing itching for psoriasis, among other uses.

The present invention is directed to natural plant extracts that have been found to improve various dermatological conditions including various disorders and diseases

### DISCLOSURE OF THE INVENTION

According to various features, characteristics and embodiments of the present invention which will become apparent as the description thereof proceeds, the present invention provides a method of treating an area of a skin suffering from at least one dermatological condition selected from psoriasis, athlete's foot, verruca, shingles, atopic dermatitis, and candida which involves the steps of:

a) providing an oil extract from leleshwa; and

b) applying the oil extract to the area of skin.

The oil extract from leleshwa can be formulated into at least one of a liquid, ointment, salve, gel, cream or foam and the oil extract can be obtained leleshwa leaves, stems, branches, bark and roots using extraction processes such as steam distillation, maceration, decoction and percolation.

### BEST MODE FOR CARRYING OUT THE INVENTION

The present invention is directed to natural plant extracts that can be used alone or combined with other ingredients as dermatological treatment compositions. In particular, the present invention involves extracts from Tarchonanthus camphorate which are obtained from a steam distillation process (direct or indirect) or other extraction process that can be used to extract oils from the leaves of Tarchonanthus camphorate plants.

The present invention is directed to the use of formulations of leleshwa which are obtained from a steam distillation process other extraction process that can used to extract essential oils from the leaves of leleshwa. Although specific reference is made herein to a steam distillation process, it is to be understood that any extraction process that allows for the separation and collection of the essential oils starting materials such as leaves, twigs, sticks, bark, roots, etc., can be used according to the present invention. Examples of other extraction processes include maceration, decoction and percolation.

The boiling points of essential oils from plants are less than the boiling point of water. Moreover, essential oils have specific gravities that are more or less than the specific gravity of water. Accordingly, essential oils can be collected over (floating on) or under water.

Essential oils are held inside aromatic plant cells. Steam can be applied to disrupt the plant cells and release the essential oils.

In a steam distillation process to remove and collect essential oils from leleshwa, leaves are collected and placed in a container through which steam is passed. Typically in steam distillation processes steam is generated in a boiler and passed through a container which is configured to support materials in a manner which will expose the materials to the steam.

As the steam contacts the leaves, the plant cells are disrupted and the essential oils that have lower boiling points than the water (i.e., lower than the temperature of the steam that contacts the leaves) are releases and formed into vapors that are carried out of the container with the steam.

The mixture flow of essential oil and steam is directed to a condenser in which the mixture if cooled to form a liquid mixture, having an aqueous phase and an oil phase which separate according to the relative specific densities. According to the present invention the aqueous phase from the steam distillation of leleshwa is discarded and the essential oils are collected and used as dermatological treatment formulations alone or together with other ingredients.

Although there are many known uses for the essential oils of leleshwa, during the course of the present invention it was unexpectedly discovered that the essential oils extracted from leleshwa were effective in treating athlete's foot, verruca, shingles, atopic dermatitis, and candida.

Athlete's foot is a skin disease caused by a fungus, usually occurring between the toes. The signs of athlete's foot, singly or combined, are drying skin, itching scaling, inflammation, and blisters. Blisters often lead to cracking of the skin. When blisters break, small raw areas of tissue are exposed, causing pain and swelling. Itching and burning may increase as the infection spreads. Athlete's foot may spread to the soles of the feet and to the toenails. It can be spread to other parts of the body, notably the groin and underarms, by those who scratch the infection and then touch themselves elsewhere. The organisms causing athlete's foot may persist for long periods. Consequently, the infection may be spread by contaminated bed sheets or clothing to other parts of the body.

Verrucas are a type of wart that are caused by a virus and are contagious. They appear in various shapes and sizes and may grow singularly or in groups. The can be painful condition and depending on the size and location. Conservative treatments include topical medicines and patches and if these treatments fail, a series of freezing methods using liquid nitrogen or electrosurgery can be used to eradicate the problem.

Shingles (herpes zoster) are an outbreak of rash or blisters on the skin that is caused by the same virus that causes chicken pox—the varicella-zoster virus. Early signs of shingles include burning or shooting pain and tingling or itching located on one side of the body or face. After several days a rash of small blisters appears on reddened skin. The piercing and unrelenting pain associated with shingles can be set off by a gentle touch or a soft breeze on the skin.

Atopic dermatitis is a chronic (long-lasting) disease that affects the skin. The word “dermatitis” means inflammation of the skin. “Atopic” refers to a group of diseases where there is often an inherited tendency to develop other allergic conditions, such as asthma and hay fever. In atopic dermatitis, the skin becomes extremely itchy. Scratching leads to redness, swelling, cracking, “weeping” clear fluid, and finally, crusting and scaling. In most cases, there are periods of time when the disease is worse (called exacerbations or flares) followed by periods when the skin improves or clears up entirely (called remissions).

Candida or Monilia is a skin rash caused by a yeast living on normal skin of 80% of all people. In most people, the presence of this yeast on the skin is not visible. In some people, for unknown reasons, the yeast grows more actively and causes a red, itchy, scaling rash. The yeast likes warm moist areas and usually grows in the skin folds under the breast, stomach, and arm pits. Treatment involves the application of an anti-yeast cream applied to the affected area twice a day followed by hydrocortisone cream.

The essential oils the leleshwa recovered from the steam distillation process can be used as a dermatological treatment formulation for athlete's foot, verruca, shingles, atopic dermatitis, and candida alone at full strength, diluted or concentrated as desired. In addition, the essential oils of the leleshwa can be combined together with other ingredients used to formulate liquids, ointments, salves, gels, creams or foams as are generally known in the art.

As noted above, the essential oils extracted from the leleshwa can be used full strength, diluted or concentrated as desired. In general, it was determined that formulations that contain as little as from about 0.01 wt. % of the essential oils of leleshwa would be effective according to the present invention, with formulations containing from about 0.01 wt. % to about 10 wt. % being useful, and formulations containing from about 0.03 wt. % to about 5 wt. %. When formulated into a liquid, ointment, salve, gel, cream or foam the compositions of the present invention may contain a wide range of additional, optional components. The CTFA Cosmetic Ingredient Handbook, Seventh Edition, 1997 and the Eighth Edition, 2000, which is incorporated by reference herein in its entirety, describes a wide variety of cosmetic and pharmaceutical ingredients commonly used in skin care compositions, which are suitable for use in the compositions of the present invention. Examples of these functional classes disclosed in this reference include: absorbents, abrasives, anticaking agents, antifoaming agents, antioxidants, binders, biological additives, buffering agents, bulking agents, chelating agents, chemical additives, colorants, cosmetic astringents, cosmetic biocides, denaturants, drug astringents, external analgesics, film formers, fragrance components, humectants, opacifying agents, pH adjusters, plasticizers, preservatives, propellants, reducing agents, skin bleaching agents, skin-conditioning agents (emollient, humectants, miscellaneous, and occlusive), skin protectants, solvents, foam boosters, hydrotropes, solubilizing agents, suspending agents (nonsurfactant), sunscreen agents, ultraviolet light absorbers, waterproofing agents, and viscosity increasing agents (aqueous and nonaqueous).

In addition to extracting the essential oils from leleshwa leaves, it is also possible to extract essential oils from the roots, bark, stems and/or branches either fresh, dried or soaked according to the present invention.

An analysis of an aqueous formulation of the essential oils leleshwa recovered from the steam distillation process if presented in Tables I and II below:

TABLE I
Volatile Organics (Capillary Column)
Tentatively Identified Compounds (TIC)

| CAS | Parameter | Result* | Units |
|---|---|---|---|
| 58037-87-9 | 4-METHYL-1-(1- | 670 | mg/kg |
| | METHYLENTHYL)- | | |
| | BICYCLOHEXANE | | |
| 99-86-5 | 1-METHYL-4-(1- | 410 | mg/kg |
| | METHYLETHYL)- | | |
| | 1,3-CYCLOHEXADIENE | | |
| 99-83-2 | ALPHA-PHELLANDRENE | 220 | mg/kg |
| 5989-54-8 | 1-METHYL-4(1- | 670 | mg/kg |
| | METHYLETHYL)CYCLOHEXENE | | |
| 586-62-9 | 1-METHYL- | 220 | mg/kg |
| | 4(METHYLETHYLIDENE)- | | |
| | CYCLOHEXENE | | |

Instrument: GC/MS
Prep: high conc. purge and trap method for organic analytes
*Estimated concentrations, TIC by GC/MS.
Compounds listed in order of retention time.

TABLE II
Semi-Volatile Organics (Base/Neutral/Acid Fractions)
Tentatively Identified Compounds (TIC)

| CAS | Parameter | Result* | Units |
|---|---|---|---|
| 2867-05-2 | 2-METHYL-5-(1-METHYLETHYL)-BICYCLOHEX-2-ENE | 2500 | mg/kg |
| 80-56-8 | ALPHA-PINENE | 92,000 | mg/kg |
| 79-92-5 | CAMPHENE | 57,000 | mg/kg |
| 104-51-8 | N-BUTYLBENZENE | 1500 | mg/kg |
| 554-61-0 | (+)-2-CARENE | 4100 | mg/kg |
| 470-82-6 | EUCALYPTOL | 140,000 | mg/kg |
| 99-85-4 | 1-METHYL-4-(1-METHYLETHYL)-1,4-CYCLOHEXADIENE | 10,000 | mg/kg |
| 562-74-3 | 4-METHYL-1-(1-METHYLETHYL)-3-CYCLOHEXADIENE | 1800 | mg/kg |
| 1632-73-1 | 1,3,3-TRIMETHYL-BICYCLO[2,2,1]HEPTAN-2-OL | 13,000 | mg/kg |
| 4948-29-2 | CIS-2-PINANOL | 2500 | mg/kg |
| 465-31-6 | 2,3,3-TRIMETHYL-BICYCLO[2,2,1]HEPTAN-2-OL | 660 | mg/kg |
| 3856-25-5 | COPAENE | 1100 | mg/kg |
| 25246-27-9 | DECAHYDRO-1,1,7-TRIMETHYLETHYL-CYCLOPROP[E]AZULENE | 1200 | mg/kg |
| 6831-16-9 | (−)-ARISTOLENE | 600 | mg/kg |
| 39029-41-9 | OCTAHYDRO-MEHTYL-NAPHTHALENE | 500 | mg/kg |
| 483-76-1 | 1,2,4A,5,6,8A-HEXAHYDRO-4,7-DIMETHYL-NAPHTHALENE | 1100 | mg/kg |
| 473-15-4 | DECAHYDRO-2-NAPHTHALENEMETHANOL | 4700 | mg/kg |

Instrument: GC/MS
*Estimated concentrations, TIC by GC/MS.
Compounds listed in order of retention time.

Although the present invention has been described with reference to particular means, materials and embodiments, from the foregoing description, one skilled in the art can easily ascertain the essential characteristics of the present invention and various changes and modifications can be made to adapt the various uses and characteristics without departing from the spirit and scope of the present invention as described above and recited in the attached claims.

What is claimed is:

1. A method of treating an area of skin suffering from at least one dermatological condition selected from psoriasis, athlete's foot, verruca, shingles, atopic dermatitis, and candida which comprises the steps of:
a) providing an oil extract from leleshwa; and
b) applying the oil extract to the area of skin.

2. A method of treating an area of skin suffering from at least one dermatological condition according to claim 1, wherein the dermatological condition is verruca.

3. A method of treating an area of skin suffering from at least one dermatological condition according to claim 1, wherein the dermatological condition is shingles.

4. A method of treating an area of skin suffering from at least one dermatological condition according to claim 1, wherein the dermatological condition is atopic dermatitis.

5. A method of treating an area of skin suffering from at least one dermatological condition according to claim 1, wherein the dermatological condition is candida.

6. A method of treating an area of skin suffering from at least one dermatological condition according to claim 1, wherein the oil extract from leleshwa is formulated into at least one of a liquid, ointment, salve, gel, cream or foam.

7. A method of treating an area of skin suffering from at least one dermatological condition according to claim 1, wherein the oil extract from leleshwa is obtained from at least one of leleshwa leaves, stems, branches, bark and roots.

8. A method of treating an area of skin suffering from at least one dermatological condition according to claim 7, wherein the oil extract from leleshwa is obtained using an extraction process selected from steam distillation, maceration, decoction and percolation.

9. A method of treating an area of skin suffering from at least one dermatological condition according to claim 8, wherein the oil extract from leleshwa is obtained using steam distillation.

10. A method of treating an area of skin suffering from at least one dermatological condition according to claim 6, wherein the formulations contain at least 0.01 wt. % of the oil extract from leleshwa.

11. A method of treating an area of skin suffering from at least one dermatological condition according to claim 10, wherein the formulations contain from at least about 0.01 wt. % to about 10 wt. % of the oil extract from leleshwa.

12. A method of treating an area of skin suffering from at least one dermatological condition according to claim 11, wherein the formulations contain from at least about 0.03 wt. % to about 5 wt. % of the oil extract from leleshwa.
